(19) United States
(12) Patent Application Publication
(10) Pub. No.: US 2023/0183637 A1
(43) Pub. Date: Jun. 15, 2023

(54) LIVING CELL SEPARATION SYSTEM

(71) Applicant: TERUMO KABUSHIKI KAISHA, Tokyo (JP)

(72) Inventors: Eri Noguchi, Ashigarakami-gun (JP); Masaki Matsumura, Ashigarakami-gun (JP)

(73) Assignee: TERUMO KABUSHIKI KAISHA, Tokyo (JP)

(21) Appl. No.: 18/165,573

(22) Filed: Feb. 7, 2023

Related U.S. Application Data
(63) Continuation of application No. PCT/JP2021/036050, filed on Sep. 30, 2021.

(30) Foreign Application Priority Data
Sep. 30, 2020 (JP) 2020-165839

(51) Int. Cl. C12M 1/00; C12N 5/077; C12M 1/34

(52) U.S. Cl. CPC C12M 47/04; C12N 5/0658; C12M 41/30; C12N 2509/10; C12N 2501/599

(57) ABSTRACT

A system that easily and stably separates various living cells from a living body-derived tissue, the system including: a mincing unit that minces the living body-derived tissue based on a parameter; a measurement unit that acquires information regarding the living body-derived tissue being minced; and an analysis unit that calculates a ratio of impurities to the living body-derived tissue being minced from the information acquired by the measurement unit. Methods for separating various living cells from a living body-derived tissue are also disclosed.

### CROSS-REFERENCES TO RELATED APPLICATIONS

This application is a continuation of International Patent Application No. PCT/JP2021/036050 filed on Sep. 30, 2021, which claims priority to Japanese Patent Application No. 2020-165839 filed on Sep. 30, 2020, the entire content of both of which is incorporated herein by reference.

### TECHNOLOGICAL FIELD

The present disclosure generally relates to a system for separating living cells from a living body-derived tissue.

### BACKGROUND DISCUSSION

In recent years, attempts have been made to transplant various cells for repair of damaged tissues and the like. For example, attempts have been made to use fetal cardiomyocytes, myoblast cells, mesenchymal stem cells, cardiac stem cells, ES cells, iPS cells, and the like for repair of myocardial tissue damaged by ischemic heart disease such as angina pectoris and myocardial infarction (Haraguchi et al., Stem Cells Transl Med. 2012 February; 1(2): 136-41).

As a part of such attempts, a cell structure formed using a scaffold and a sheet-shaped cell culture in which cells are formed in a sheet shape have been developed (Sawa et al., Surg Today. 2012 January; 42(2): 181-4).

For the application of the sheet-shaped cell culture to treatment, studies have been made on the use of a cultured epidermal sheet for skin damage caused by burns or the like, the use of a corneal epithelial sheet-shaped cell culture for corneal damage, the use of an oral mucosa sheet-shaped cell culture for endoscopic resection of esophageal cancer, and the like, and some of them have entered the stage of clinical application.

Myoblast cells used for such treatment are usually obtained by separating CD 56 positive cells such as myoblast cells from skeletal muscle tissue to be transplanted. As a measure to increase a ratio of the CD 56 positive cells contained in cells separated from the skeletal muscle tissue, for example, a method is known that includes a first process involving immersing skeletal muscle tissue in a first protease solution for a prescribed time and then discarding the obtained first enzyme-treated solution and a second process involving immersing the skeletal muscle tissue resulting from the first process in a second protease solution for a prescribed time and then recovering the cells contained in the second enzyme-treated solution (JP 2007-89442 A).

### SUMMARY

In order to separate living cells from living body-derived tissue as described above, various separation methods and separation conditions have been devised. However, there are various cells constituting various living tissues, and among them, stem cells and precursor cells have a low proportion in the living tissue, and many exist in a special niche. For example, skeletal muscle tissue is composed of muscle fibers, and the parenchyma of the muscle fibers is multi-nucleus cells surrounded by a plasma membrane. However, CD 56 positive cells such as myoblast cells, which are precursor cells of the muscle fibers, are localized only between the basement membrane and the plasma membrane of the muscle fibers. Therefore, in order to separate these CD 56 positive cells from the skeletal muscle tissue of a subject, it is necessary to disrupt the skeletal muscle tissue to such an extent that the basement membrane of the muscle fibers is loosened and the CD 56 positive cells are not destroyed.

So far, separation of CD 56 positive cells from skeletal muscle tissue has been mainly performed by manual mincing and enzymatic degradation. The operation of disrupting muscle fibers by the mincing process is complicated and takes a long time, and the determination depends on intuition of the worker, so that the number of recovered living cells among workers varies. As described above, a method of easily and stably separating various living cells from various living body-derived tissues has not yet been found.

As a result of intensive studies to solve the above-described problems, the present inventors have found that it is possible to stably separate living cells from living body-derived tissue by acquiring information on the living body-derived tissue and appropriately calculating a ratio of impurities to living cells in the living body-derived tissue during the mincing process. As a result of further studies based on such findings, the present inventors have found that the ratio of impurities to living cells in the living body-derived tissue can be mechanically calculated, and the overall work or a part thereof can be automated.

That is, the present disclosure relates to the following:

[1] A system that separates living cells from a living body-derived tissue, the system including:
a mincing unit that minces the living body-derived tissue;
a measurement unit that acquires information on a parameter of the living body-derived tissue being minced; and
an analysis unit that calculates a ratio of impurities to living cells in the living body-derived tissue being minced from the information acquired by the measurement unit.

The system according to [1], in which the analysis unit further determines a separation state from the ratio of the impurities.

The system according to [1] or [2], further including a learning unit that extracts excess or deficiency of the parameter based on information from the analysis unit.

The system according to any one of [1] to [3], further including an update unit that updates the parameter based on excess or deficiency of the parameter extracted by a learning unit.

[5] The system according to any one of [1] to [4], further including a stirring unit that stirs the minced living body-derived tissue.

[6] The system according to any one of [1] to [5], further including a removal unit that removes impurities from the minced living body-derived tissue based on information from the analysis unit.

[7] The system according to any one of [1] to [6], in which the parameter includes at least one of a number of mincing, an angle of mincing, a force applied to mincing, and a location of mincing.

[8] The system of any one of [1] to [7], in which the information regarding the living body-derived tissue being minced includes at least one of a color of impurities, a size of impurities, and a force applied to mincing.

[9] The system of any one of [1] to [8], in which the living cells are myoblasts.

According to the system of the present disclosure, the operation of separating living cells from living body-derived tissue can be performed simply, reliably, and automatically, and not only variations in the number of recovered living cells among workers can be eliminated, but also labor of the workers can be greatly reduced.

Another aspect of the present disclosure relates to a method for separating living cells from living body-derived tissue, the method including at least one of: mincing the living body-derived tissue in a mincing unit; acquiring information on a parameter of the minced living body-derived tissue with a measurement unit; and calculating a ratio of impurities in the minced living body-derived tissue from the information acquired by the measurement unit with an analysis unit.

### BRIEF DESCRIPTION OF THE DRAWINGS

FIG. 1 illustrates a flow diagram of an exemplary embodiment of a system disclosed herein that determines if separation of the living cells is completed.

FIG. 2 illustrates a flow diagram of an exemplary embodiment of a system described herein that determines if separation of the living cells is completed and removes impurities from the mincing unit until separation is completed.

FIG. 3 illustrates an example of components of a system that separates living cells from a living body-derived tissue.

### DETAILED DESCRIPTION

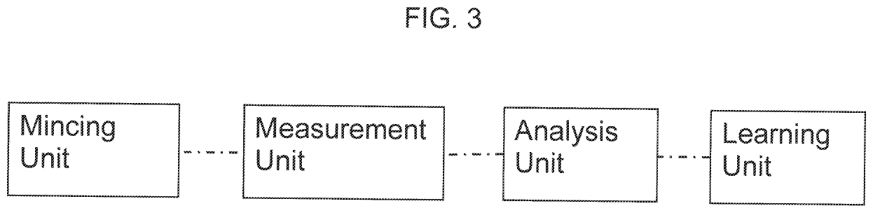

The present disclosure relates to a system that separates living cells from a living body-derived tissue, the system including, as generally shown in FIG. 3, a mincing unit that minces the living body-derived tissue based on a parameter; a measurement unit that acquires information regarding the living body-derived tissue being minced; and an analysis unit that calculates a ratio of impurities to the living body-derived tissue being minced from the information acquired by the measurement unit.

In the present disclosure, the living body-derived tissue is not particularly limited as long as it is derived from a living body, and is, for example, muscle tissue, fat tissue, skin tissue, cartilage tissue, tendon tissue, ligament tissue, interstitium, vascular tissue, brain tissue, circulatory system tissue, digestive system tissue, metabolic system tissue, lymphatic system tissue, bone marrow tissue, blood, or the like, preferably muscle tissue, fat tissue, bone marrow tissue, blood, and more preferably skeletal muscle tissue.

The living cell in the present disclosure can include any living cell separated from the living body-derived tissue. Non-limiting examples thereof include cardiomyocytes, fibroblast cells, epithelial cells, endothelial cells, hepatocytes, pancreatic cells, renal cells, adrenal cells, periodontal ligament cells, gingival cells, periosteal cells, skin cells, synoviocytes, chondrocytes, and the like, and stem cells (for example, myoblasts (for example, myoblast cells) (Myoblasts include satellite myocytes), mesenchymal stem cells (for example, those derived from bone marrow, adipose tissue, peripheral blood, skin, hair roots, muscle tissue, endometrium, placenta, cord blood, and the like), tissue stem cells such as cardiac stem cells, embryonic stem cells, etc.). The living cell in the present disclosure may be a cell that is in direct contact with a plurality of membranes or localized so as to be surrounded by the membranes so as to be adjacent to the plurality of membranes between the plurality of membranes in contact with each other in a plane. In the present disclosure, examples of the living cells preferably include CD 56 positive cells in skeletal muscle tissue such as myoblast cells, or mesenchymal stem cells derived from bone marrow, adipose tissue, and peripheral blood.

In the present disclosure, cells, tissues, and the like other than the living cells that are intended to be collected from a living body-derived tissue are referred to as impurities. For example, when myoblasts and the like are separated from skeletal muscle tissue, white tissue (tendons, blood vessels, fats, etc.) and fibroblast cells become impurities.

The living body-derived tissue used in the present disclosure can be derived from any organism. Such organisms include, but are not limited to, humans, non-human primates, rodents (mice, rats, hamsters, guinea pigs, etc.), dogs, cats, pigs, horses, cows, goats, sheep, and the like. When cells separated from the living body-derived tissue are used for transplantation, the living body-derived tissue used in the present disclosure can avoid rejection by using autologous cells separated using the living body-derived tissue collected from the subject (recipient) himself/herself. However, it is also possible to use heterologous cells or homologous non-autologous cells separated using heterologous or homologous non-autologous living body-derived tissues.

In the present disclosure, the “mincing unit” is a unit that minces a living body-derived tissue based on a parameter. The mincing unit is, for example, a robot that operates on the basis of setting of a parameter, in which a tip has a knife shape, and minces a living body-derived tissue. The parameters include, for example, the number of times of mincing, an angle of mincing, a force applied to mincing, and a location of mincing. The living body-derived tissue may be placed in a range where a tip end part of the mincing unit reaches, and may be placed in a commercially available cell culture container, for example, a petri dish, a tube, a flask, or the like. Furthermore, for example, the mincing unit includes a sensor for grasping the living body-derived tissue in the container, so that a location of mincing can be determined, but the container containing the living body-derived tissue may be integrated with the mincing unit so that the mincing unit can easily determine the location of mincing.

In the present disclosure, the “measurement unit” is a unit that acquires information regarding a living body-derived tissue being minced. Examples of the information regarding the living body-derived tissue include a color of impurities, a size of impurities, and a force applied to mincing, and information suitable for calculating a ratio of the impurity to the living body-derived tissue can be acquired. As a method of calculating the ratio of impurities to living body-derived tissue, any known method can be used, and for example, an image analysis method, a force detection method, or the like can be used.

In the image analysis method, the acquired image is analyzed to measure the size and color of impurities in the image, and the ratio of impurities in the living body-derived tissue is calculated. For example, the size and color of impurities are extracted from an image acquired by a CCD camera or a device combining a microscope and a CCD camera, and the ratio of the impurities to the living body-derived tissue is calculated. The calculated ratio may be based on the entire living body-derived tissue, or may be based on a part thereof, for example, per unit area. Therefore, the acquired image may be of the entire living body-derived tissue or a part thereof. A CCD camera or a device combining a microscope and a CCD camera may represent an example of a measurement unit that acquires information regarding a living body-derived tissue being minced.

For example, when myoblasts are separated from skeletal muscle tissue, the impurities are white tissue (tendons, blood vessels, fats, etc.). In such an aspect, the impurities are finer in the mincing process, but finally remain in a form larger than a part of the target living cell, so that the part of the target living cell and the impurities can be distinguished in the image, and thus the ratio of the impurities to the living body-derived tissue can be calculated in the image analysis method. Furthermore, since the impurities are whiter in color than the part of the target living cell, and the part of the target living cell and the impurities can be distinguished in the image, the ratio of the impurities to the living body-derived tissue can be calculated by the image analysis method.

A force detection method detects a force applied to the mincing unit when the mincing unit minces a living body-derived tissue. A force applied to the mincing unit differs between when only a part of the target living cell of the living body-derived cell is minced and when a part in which impurities are mixed is minced. Therefore, for example, the ratio of impurities to the living body-derived tissue is calculated by measuring the applied force itself or the number of times the applied force is different when the mincing is repeated a certain number of times using a pressure sensor attached to the mincing unit or a device in which a counter and a pressure sensor are combined. For example, when myoblasts are separated from skeletal muscle tissue, the impurities are white tissue (tendons, blood vessels, fats, etc.). In such an aspect, although the impurities are finer in the mincing process, the impurities remain larger than the part of the target living cells in the end, so that the force applied to the mincing unit is larger than that when the part of the target living cells is minced, and it is possible to distinguish whether a part where the mincing unit minces is the part of the target living cells or the impurities from the force. Therefore, the ratio of the impurities to the living body-derived tissue can be calculated by the force detection method.

In the present disclosure, the “analysis unit” is a unit that receives information from the measurement unit and calculates the ratio of the impurities to the living body-derived tissue being minced. The analysis unit may compare the calculated value of the ratio of the impurities with a preset threshold value, and determine a separation state of the living body-derived tissue based on the comparison result. In the present disclosure, the separation includes separating the target living cells from the living body-derived tissue that is a large mass, exposing impurities integrated inside the living body-derived tissue to a surface by subjecting the living body-derived tissue that is a large mass to the mincing process, and removing the exposed impurities. The comparison with the threshold value set in advance includes that as the living body-derived tissue leaving a large mass is minced, an amount of the impurities exposed on the surface increases and exceeds the threshold value set in advance, or that by removing the impurities, the calculated ratio of the impurities decreases and falls below the threshold value set in advance.

The analysis unit includes at least a processor (central processing unit) that receives the information from the measurement unit, calculates the ratio of the impurities to the living body-derived tissue being minced from the information, and compares the calculated value with the threshold value to determine the separation state, but may further include a storage unit, a control unit, an input unit, and an output unit. The storage unit is a unit that stores the information from the measurement unit, calculated values, determination of the separation state, and the like, and includes various electronic storage media, for example, a semiconductor memory, a hard disk, and the like. The control unit is a unit that transmits a signal to the mincing unit or the like based on the calculated value or the determination of the separation state, and includes a signal generation circuit or the like. The input unit is a unit to which an operator of the system of the present disclosure, another unit of the system, or another system inputs information such as a threshold value as necessary, and includes various input interfaces, for example, a unit (electric wire, optical fiber, connector, wireless communication device, and the like) that receives a signal such as electricity and light from another system, a button, a keyboard, a touch panel, and the like. The output unit is a unit that emits a predetermined signal on the basis of the calculated value, the determination of the separation state, and the like, and includes various output interfaces, for example, a unit (electric wire, optical fiber, connector, wireless communication device, and the like) that transmits a signal such as electricity or light to another unit, a monitor, a printer, an indicator lamp, a buzzer, a speech synthesizer, and the like. The input and output units may be integrated as an input/output interface, including an input interface and an output interface, and a general-purpose computer may be utilized for this purpose.

The system of the present disclosure can further include a learning unit. In the present disclosure, the “learning unit” is a unit that extracts excess or deficiency of a parameter on the basis of information from the analysis unit. The learning unit extracts excess or deficiency of the parameter by associating the parameter of the mincing unit with an output from the analysis unit regarding the separation state determined by the analysis unit, or a part recognized as impurities or a part recognized as not being impurities or a part recognized as containing a large amount of impurities by the analysis unit in the analysis process. For example, when the living body-derived tissue starts to be minced and the living body-derived tissue still remains as a large mass, the learning unit can determine that a part recognized as having no impurities or not containing a large amount of impurities does not appear on the surface and thus the number of times of mincing is insufficient. Furthermore, in a case where the analysis unit recognizes that there are impurities in only a part of the living body-derived tissue, it is possible to make a determination so as to mince a position other than the part.

The learning unit may further include a learning storage unit. The learning storage unit is a unit that accumulates and stores a parameter of the mincing unit and information of the parameter from the analysis unit, and includes various electronic storage media similarly to the storage unit. The learning unit may extract excess or deficiency of the parameter with higher accuracy with reference to the data stored in the learning storage unit. Furthermore, the learning unit may further include a learning input unit and a learning output unit. The learning input unit is a unit that inputs information that enables an operator of the system of the present disclosure, another unit of the system, or another system to perform more efficient and highly accurate learning than in a mincing environment or the like as necessary, and the learning output unit is a unit that emits a predetermined signal on the basis of the extracted excess or deficiency of the parameter. The learning input unit and the learning output unit may include interfaces similar to the input unit and the output unit, respectively.

The system of the present disclosure may further include an update unit. In the present disclosure, the “update unit” is a unit that updates a parameter on the basis of excess or deficiency of the parameter extracted by the learning unit. The update unit receives information from the learning unit, creates a new parameter reflecting the information, and updates the parameter of the mincing unit to the created parameter. The update unit may include an output interface for outputting to the mincing unit, or may be integrated with the mincing unit. Alternatively, the update unit is integrated with the learning unit, and may not receive the information from the learning unit, but may create a new parameter and update the parameter of the mincing unit on the basis of the information from the analysis unit.

The system of the present disclosure may further include a stirring unit. In the present disclosure, the “stirring unit” is a unit that stirs the minced living body-derived tissue. The stirring unit may be, for example, a unit that supports a container containing a living body-derived tissue from below and may stir the container by shaking, rotating, or the like, or may be a robot having a tip in a rod or spatula shape and stir the container by moving a tip portion of the robot, or may be a combination thereof. The stirring unit has a function of uniformizing a minced state of the living body-derived tissue, and when the stirring unit stirs the living body-derived tissue, the separation state of impurities becomes equal in any part of the living body-derived tissue. Therefore, the measurement unit can acquire information sufficient for calculating the ratio of the impurities to the minced living body-derived tissue by acquiring information on a part of the living body-derived tissue.

The system of the present disclosure may further include a removal unit. In the present disclosure, the “removal unit” is a unit that removes the impurities from the minced living body-derived tissue based on the information from the analysis unit. The removal unit is, for example, a robot that removes the impurities from the living body-derived tissue and has a needle or tweezers shaped tip. The removal unit removes the impurities, for example, on the basis of an output from the analysis unit regarding a part recognized as impurities, a part recognized as containing a large amount of impurities, or the like by the analysis unit in the analysis process. The output may reach the removal unit via an output unit associated with the analysis unit. In one aspect, the removal unit may include an output interface that outputs to the mincing unit for further mincing after the removal operation.

As described above, the components constituting the system of the present disclosure can be arranged in various manners as long as a predetermined object can be achieved, and can be combined or integrated as necessary.

Hereinafter, the system of the present disclosure will be described in more detail with reference to the drawings, which show exemplary embodiments that are in accordance with the present disclosure. The present disclosure is not limited to these exemplary embodiments.

In one aspect, the separation of living cells from a living tissue-derived tissue in the system of the present disclosure is roughly divided into two stages. When the mincing process of the living body-derived tissue is started, the living body-derived tissue remains as a large mass, and impurities are not exposed on the surface. At this stage, the exposure of the impurities to the surface increases as the mincing process proceeds. Next, when the living body-derived tissue is minced to such an extent that no large mass is observed, it is newly added to remove the recognized impurities. At this stage, the recognized impurities decrease as the mincing process and the removal of the impurities proceed.

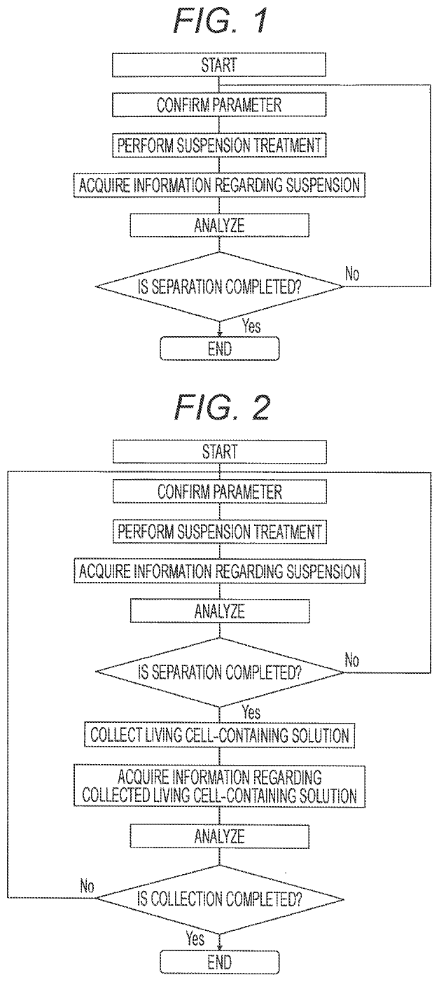

FIG. 1 illustrates a flow diagram of separating living cells from a living body-derived tissue when the mincing process of the living body-derived tissue starts in one aspect of the system of the present disclosure. In the present aspect, when the system receives a separation start instruction, the mincing unit starts the mincing process according to the input parameters. The measurement unit acquires information regarding the living body-derived tissue being minced in the mincing process of the mincing unit, for example, a size of impurities, a color of impurities, a force applied to the mincing unit, and the like. In the analysis, a ratio of the impurities to the living body-derived tissue being minced is calculated from the information on the living body-derived tissue being minced. When the mincing process is sufficiently performed, a large mass is not recognized from the living body-derived tissue, and the impurities present inside the living body-derived tissue are also exposed to a surface, so that the ratio of the impurities to the living body-derived tissue increases. In a case where a large mass remains in the living body-derived tissue, the ratio of the impurities to the living body-derived tissue is still low, and by further subjecting the living body-derived tissue to the mincing process, a large mass is not observed in the living body-derived tissue, and the ratio of the impurities to the living body-derived tissue increases. In one aspect of the present disclosure, the analysis unit can determine that the separation in this aspect is completed when the ratio of the impurities to the living body-derived tissue exceeds a preset threshold value. When the separation is completed, the system of the present disclosure can output a separation complete signal to end the flow. In a case where the analysis unit determines that the separation is not completed, the analysis unit outputs a signal to the mincing unit, and the living body-derived tissue can be further minced by the mincing unit. In this case, the parameter of the mincing can be changed on the basis of the information from the analysis unit. The operator of the system may change the parameter by inputting a parameter into the mincing unit. In one aspect, the system of the present disclosure may further include a learning unit and an update unit, a signal may be output from the analysis unit to the learning unit, and in these units, a parameter may be changed and applied (updated) to the mincing unit.

FIG. 2 illustrates a flow diagram of separating living cells from a living body-derived tissue when the living body-derived tissue is minced to an extent that no large mass is observed in one aspect of the system of the present disclosure. Also in the present aspect, similarly to the flow in FIG. 1, when the system receives the instruction to start separation, the mincing unit starts the mincing process according to a parameter that is input to the mincing unit, the measurement unit acquires information regarding the living body-derived tissue being minced in the mincing process of the mincing unit, for example, a size of impurities, a color of impurities, a force applied to the mincing unit, and the like, and the analysis unit calculates a ratio of the impurities to the living body-derived tissue being minced from the information regarding the living body-derived tissue being minced. In one aspect of the present disclosure, the analysis unit can determine that the separation in this aspect is completed when the ratio of the impurities to the living body-derived tissue falls below a preset threshold value. When the separation is completed, the system of the present invention can output a separation complete signal to end the flow. In a case where the analysis unit determines that the separation is not completed, the analysis unit can output a signal to a removal unit to remove the impurities in the living body-derived tissue by the removal unit. The removal unit outputs a signal to the mincing unit after removing the impurities, and the living body-derived tissue from which the impurities have been removed can be further minced in order to expose the impurities hidden in the tissue. For the living body-derived tissue subjected to the further mincing, the measurement unit acquires information on the living body-derived tissue being minced, and the analysis unit recalculates the ratio of the impurities to the living body-derived tissue being minced from the information regarding the living body-derived tissue being minced, and determines the completion of the separation again. In this way, the removal of the impurities and the mincing can be repeated until the separation is completed. Furthermore, also in the present aspect, the mincing parameter can be changed on the basis of the information from the analysis unit, and such a change may be input by the operator of the system or may be made by the learning unit and the update unit.

Although one aspect of the system of the present disclosure has been described above, it should be understood that various aspects other than the above are possible. Therefore, various aspects obtained by modifying the above aspects without departing from the spirit of the present disclosure are also included in the scope of the present disclosure, and such modifications are understandable to those skilled in the art.

What is claimed is:

1. A method for separating living cells from living body-derived tissue, the method comprising:
mincing the living body-derived tissue to produce minced living body-derived tissue;
acquiring information about a parameter of the minced living body-derived tissue; and
calculating a ratio of impurities to living cells in the minced living body-derived tissue using the information acquired about the parameter.

2. The method according to claim 1, wherein the living body-derived tissue is derived from muscle tissue, fat tissue, skin tissue, cartilage tissue, tendon tissue, ligament tissue, interstitium, vascular tissue, brain tissue, circulatory system tissue, digestive system tissue, metabolic system tissue, lymphatic system tissue, bone marrow tissue or blood.

3. The method according to claim 1, wherein the living cells are cardiomyocytes, fibroblast cells, epithelial cells, endothelial cells, hepatocytes, pancreatic cells, renal cells, adrenal cells, periodontal ligament cells, gingival cells, periosteal cells, skin cells, synoviocytes, chondrocytes, or stem cells.

4. The method according to claim 1, wherein the living cells include CD 56 positive cells.

5. The method according to claim 1, wherein the living body-derived tissue is derived from a human.

6. A system that separates living cells from a living body-derived tissue, the system comprising:
a mincing unit that minces the living body-derived tissue;
a measurement unit that acquires information on a parameter of the living body-derived tissue that is subjected to the mincing by the mincing unit; and
an analysis unit that calculates a ratio of impurities to living cells in the living body-derived tissue being subjected to the mincing by the mincing unit based on the information acquired by the measurement unit.

7. The system according to claim 6, wherein the analysis unit further determines a separation state from the ratio of the impurities.

8. The system according to claim 6, further comprising a stirring unit that stirs the minced living body-derived tissue.

9. The system according to claim 6, further comprising a removal unit that removes impurities from the minced living body-derived tissue based on information from the analysis unit.

10. The system according to claim 6, wherein the parameter includes at least one of a number of mincing, an angle of mincing, a force applied to mincing, and/or a location of mincing.

11. The system according to claim 6, wherein the information regarding the living body-derived tissue being minced includes at least one of a color of impurities, a size of impurities, and/or a force applied to mincing.

12. The system according to claim 6, wherein the living cells are myoblasts.

13. The system according to claim 6, wherein the mincing unit includes a sensor for grasping the living body-derived tissue in a container so that a location of mincing can be determined.

14. The system according to claim 15, wherein the container is a petri dish, a tube or a flask.

15. The system according to claim 6, wherein the mincing unit includes a robot that operates on the basis of setting of the parameter and contains a tip having a knife shape that minces the living body-derived tissue.
